(19) United States
(12) Patent Application Publication
(10) Pub. No.: US 2024/0412875 A1
(43) Pub. Date: Dec. 12, 2024

(54) MULTIMODAL METHOD FOR DETECTING A CHANGE IN A PATIENT'S PHYSIOLOGICAL CONDITION, AND DEVICE FOR MONITORING A PATIENT SO AS TO IMPLEMENT SUCH A METHOD

(71) Applicants: INSTITUT NATIONAL DE LA SANTÉ ET DE LA RECHERCHE MÉDICALE, Paris (FR); INSTITUT DU CERVEAU ET DE LA MOELLE EPINIÈRE, Paris (FR); CENTRE NATIONAL DE LA RECHERCHE SCIENTIFIQUE, Paris (FR); ASSISTANCE PUBLIQUE - HÔPITAUX DE PARIS, Paris (FR); SORBONNE UNIVERSITÉ, Paris (FR)

(72) Inventors: Thomas SIMILOWSKI, Paris CEDEX 13 (FR); Xavier NAVARRO-SUNE, Paris CEDEX 13 (FR); Mario CHAVEZ, Paris CEDEX 13 (FR); Mathieu RAUX, Paris CEDEX 13 (FR)

(21) Appl. No.: 18/699,396

(22) PCT Filed: Oct. 8, 2021
(86) PCT No.: PCT/FR2021/051756

§ 371 Date: Apr. 8, 2024

(51) Int. Cl. G16H 50/30; A61B 5/00; A61B 5/31; A61B 5/369; G16H 40/63

(52) U.S. Cl. CPC G16H 50/30; A61B 5/31; A61B 5/369; A61B 5/7221; G16H 40/63

(57) ABSTRACT

The invention relates to a method for detecting a change in a patient's physiological condition with respect to a reference physiological condition. The method is based on a multimodal analysis that involves measurements of electroencephalography signals Sᵢ from the patient and at least one other physiological signal S_{N+1}(N≥1) from the patient. Based on these measurements, the invention proposes calculating distances di(m) associated with the electroencephalography measurements and with the measurements of the other physiological signals and fusing these data, the data having previously undergone a certain number of mathematical processing operations. In the context of the invention, the fused data may be assigned a weighting coefficient chosen according to one or more a priori or a posteriori criteria.

### TECHNICAL FIELD OF THE INVENTION

The invention relates to the methods for detecting a change in a patient's physiological condition relative to a reference physiological condition. The invention also relates to a device for monitoring a patient for implementing such a method.

The invention is part of an effort to improve the characterisation of the respiratory discomfort. The invention thus offers a solution for improving the characterisation of the changes in physiological condition, particularly changes in the respiratory condition of an anaesthesia-resuscitation patient.

### TECHNICAL BACKGROUND

The assessment of patient's respiratory condition is carried out routinely as part of a general approach to diagnosing the patient's condition. This assessment is referred to as “pre-operative” when it is carried out before the operation, “intraoperative” when it is carried out during the operation and “post-operative” when it is carried out after the operation on the patient. It is of critical importance in detecting any dyspnoea occurring in the patient during these different clinical phases. Of course, there are many other applications that require a monitoring of a patient's respiratory condition, such as detecting breathing disorders during sleep.

Human respiratory function is autonomously regulated in the brain stem. However, in the event of a disturbance in the patient's normal ventilatory flow rate, often reflecting a respiratory discomfort for the patient, certain cortical regions may be urged. These cortical activations, responsible for a change in the patient's respiratory condition, are designed to recruit the auxiliary respiratory muscles in order to compensate for any inadequate ventilatory flow rate. The term “muscle recruitment” is used because the muscle action is triggered by the nerve signals associated with the cortical activation. A disturbance in the patient's normal ventilatory flow rate therefore leads to a series of changes designed to regulate various physiological constants, referred to as homeostatic changes, which can be measured by biomedical signals.

These include, for example, the presence of a specific signature in the patient's electroencephalogram (EEG), an increase in the electromyographic (EMG) activity of the patient's ventilatory muscles and/or a change in the dynamics of the respiratory tracings. It may also be a change in the patient's cardiac activity—measured by electrocardiogram (ECG)—since a change in the patient's respiratory condition can also trigger a stress response. Similarly, a change can be observed in the patient's electrodermal response.

The prior art is limited to either analysing and processing a single type of physiological data (monomodal) to characterise the changes in the patient's physiological condition, or, when several types of physiological data (multimodal) are analysed to characterise the changes in the patient's physiological condition, to processing them separately.

Numerous methods for detecting one or more changes in a patient's physiological condition are known to the prior art, but they are based solely on the analysis and the use of a single type of physiological signal.

Such methods are known, for example, from the documents U.S. Pat. No. 5,820,560 A, US2010252038 A1 and WO2013164462 A1. The methods described are based, depending on the case, solely on data relating to the electromyographic activity of the patient's respiratory muscles or on the patient's electroencephalographic data. The monomodal methods (based on information from a single type of signal) described in these documents require a step to confirm that a change in the observed signal corresponds to a real change in the patient's physiological condition, according to the measured signal. This requires the ability to associate the time of occurrence of a physiological change in the measured signal or signals with the physiological event itself, bearing in mind that the measured signal or signals in question may be very noisy. The aforementioned confirmation step may therefore require the use of several different sensors or any other means of measurement for the analysis of the same type of homeostatic modification in order to identify the moment of the physiological event more precisely.

In addition, in order to confirm a real change in the patient's physiological condition, it may be necessary with the methods of the prior art to apply complex processing to the recorded signals.

This is the case, for example, in the document U.S. Pat. No. 5,820,560 A, in which a series of processes is required so as to extract the relevant information from electromyograms. This document report itself (Col. 5, I. 51 and following) the difficulty of keeping the noise level in the signals as low and constant as possible. Signal amplifiers and signal converters are therefore used to compensate for the effects of the diaphragm movement. A large number of sensors are also required.

The solutions proposed in these single-mode methods do not sufficiently limit the occurrence of false alarms from the respiratory assistance device and/or are too complex to implement. Another disadvantage of single-mode methods is that they intrinsically only allow observation of one physiological parameter of the patient, which does not allow characterisation of the patient's general condition.

Multimodal methods combining several physiological signals to detect a change in the patient's physiological condition have therefore also been proposed.

An example of such a method is described in the document WO 2013/140229 A1. This document discloses a method for regulating a mechanical respiratory assistance device. According to the approach used in this document, the patient's respiratory assistance is regulated as a function of the evolution of the Paw/Eadi ratio, where the Paw parameter is the value of the signal due to the ventilatory pressure exerted, while the Eadi parameter is the value of the electrical signal caused by the activity of the patient's diaphragm. The parameter Paw represents the patient's spontaneous respiratory activity and is therefore associated with the muscle pressure Pmusc. The Eadi parameter represents the respiratory neuronal activity that causes the movements of the diaphragm. The Paw/Eadi ratio is used to regulate the ventilatory assistance provided to the patient. This ratio allows to deduce the proportion in which the patient contributes to breathing in relation to the total breathing generated by himself and by the ventilatory assistance device. The aim is therefore to provide an estimate of the measure to which the patient intervenes in breathing in order to adjust the respiratory assistance. The measure to which the patient contributes to breathing cannot be used to characterise a change in its physiological condition. In fact, a certain number of the patient's respiratory movements can come from the patient himself without this being linked to a respiratory discomfort. In addition, this multimodal method does not use the correlations between the two aforementioned physiological parameters, but extracts a single piece of information from the measurements of the physiological signals associated with these two physiological parameters.

The document US 2004/0254493 describes a method for detecting changes in a patient's physiological condition based on the analysis of a plurality of physiological signals. This document focuses more specifically on the detection of breathing disorders occurring during a patient's sleep. The detection method is based on the analysis of changes in electroencephalographic signals during the individual's respiratory cycle. In addition to the analysis of electroencephalograms, this detection method also involves the analysis of other signals characterising the patient's physiological condition, such as electro-oculograms, electromyograms, oral and nasal airflow measurements, and so on. However, the method merely provides for the simultaneous recording of the various signals without any processing to take advantage of any complementarities associated with the joint acquisition of these various signals. In fact, only data from electroencephalographic signals are processed in any particular way. The practitioner therefore receives raw data which he has to interpret himself. The method is therefore not suitable for real-time modification of the patient's respiratory assistance. This is probably due to the fact that the monitoring of the respiratory condition of patients with disorders that occur during the sleep phase is generally aimed at making a diagnosis, with treatment taking place afterwards.

### SUMMARY OF THE INVENTION

The invention allows to overcome the disadvantages of the methods known in the prior art by exploiting and correlating data from different physiological signals characterising the patient's condition in order to better detect and characterise a change in the patient's condition.

In this respect, the invention relates to a method for detecting a change in the physiological condition of a patient relative to a reference physiological condition associated with a reference matrix X_{ref} with signals X_{ref,i}(i=[1 . . . N+1], N integer) and a reference period W₀, the method implementing the following steps in a loop:
A) in M time segments m of an observation window, performing measurements of electroencephalographic signals S₁ from the patient along n paths and at p times to generate M measurement matrices X_{1,m}(m∈[1 . . . M], M integer) each comprising n*p samples and simultaneously performing measurements of at least N other physiological signals S_{N+1}(N≥1) from the patient, different from the electroencephalographic signals S₁, to generate N other measurement matrices X_{i=2 . . . N+1,m},
C) for each time segment m of the reference period W₀, determining distances dᵢ(m) between each measured signalX_{i,m} and the component X_{ref,i} of the reference period,
D) transforming the distances dᵢ(m) calculated in step C) using a log d(m) function,
E) choosing the precision of the values obtained at the end of step D by selecting a reference quantile u₀ from all the values of the reference period W₀ and associating with each distance dᵢ(m) a scalar variable p_{i=1, . . . , N+1} (0<p_{i=1, . . . ,N+1}<1) depending on the reference quantile u,
F) merging the data obtained in step E) by performing a weighted sum of the variables p_{i=1, . . . , N+1} to obtain the resulting distances d_{fusion}(m),
G) determining a deviation e(m) from the physiological reference condition as a function of the distances d_{fusion}(m).

The invention thus proposes a multimodal method for characterising a patient's physiological condition, in which data from different types of measured physiological signals are processed and merged so as to deduce a change in the patient's physiological condition. The invention thus goes beyond the methods of the prior art and allows to obtain reduced variables resulting from the combination of the data acquired by the various signals. The information obtained is therefore more qualitative, more reliable and more accurate than that available in the prior art. Analysis of various physiological signals S₁, . . . , S_{N+1} provides a better characterisation of the patient's condition. Merging the data from these different signals allows to reduce, or even eliminate, non-qualitative data segments, minimise the weight of artefacts in the final data, which results in a better quality information and therefore reduces the false alarms from the medical assistance device. The invention thus offers a real-time monitoring solution that automatically acts on the respiratory assistance device and allows to reduce the unnecessary practitioner intervention on the patient.

According to various characteristics of the invention which may be taken together or separately:
in a step B) the electroencephalographic signals S₁ are centered and filtered in Q predetermined frequency bands to obtain MxQ filtered measurements matrices X_{1,m,q} (q∈[1 . . . Q]) and MxQ normalised spatial covariance matrices are determined by the formula:

$${C}_{m , q} = \frac{{X}_{1 , m , q} {X}_{1 , m , q}^{T}}{trace ( {X}_{1 , m , q} {X}_{1 , m , q}^{T} )} ;$$

in step C), Riemannian distances d_{i,r}(m) are determined between each normalised spatial covariance matrix C_{m,q} and the components X_{ref,i}, of the reference matrix associated with the electroencephalographic measurements;
in step C), statistical distances d_{i,s}(m) are determined between one or more measured signals X_{i,m}, other than the electroencephalographic signals, and the associated component or components X_{ref,i} of the reference period;
d_{fusion}(m) is calculated according to the following relationship:

$${d}_{fusion} ( m ) = {\sum }_{i} {λ}_{i} {p}_{i = 1 , … , N + 1} ( m ) , with$$

$${p}_{i = 1 , … , N + 1} ( m ) = \frac{1}{1 + {e}^{{u}_{0}} - log ( {d}_{i = 1 , … , N + 1} ( m ) )}$$

where λᵢ is the weight assigned to each variable p_{i=1, . . . ,N+1}, the weights λᵢ satisfying Σᵢλᵢ=1, u₀ is the reference quantile, d_{1,q}(m) are the Riemannian distances and d_{i>1}(m) are the statistical distances;
the weights λᵢ are selected according to the quality of the signal Sᵢ with which they are associated or according to the importance of the signal Sᵢ with which they are associated for determining a change in the patient's physiological condition;
a step of filtering the resulting distances d_{fusion}(m) is implemented prior to step F);
the filtering consists of a smoothing obtained by a moving average over the last L segments of the observation window (1<L<M) applied to the resulting distances d_{fusion}(m), said distance d_{fusion}(m) then being calculated according to the following relationship:

$${d}_{fusion}^{L} ( m ) = \frac{1}{L} {\sum }_{i = 0}^{L - 1} {d}_{fusion} ( m - i )$$

the last L segments are chosen so that the moving average is calculated taking into account the signals measured only with a delay of between 10 seconds and 1 minute, preferably between 30 seconds and 1 minute, relative to the start of the observation window;
said other physiological signals Sᵢ are cardiac activity and/or respiratory activity and/or muscular activity and/or a movement-related signal;
the method further comprising a step during which each of the deviations e(m) is compared with a determined threshold Θ.

The invention further relates to a patient monitoring device for implementing a method as previously described, the device comprising:
electroencephalographic signal measuring means for measuring the patient's electroencephalographic signals,
means for measuring the cardiac activity and/or the respiratory activity and/or the muscular activity and/or a movement,
real-time signal processing means, said means comprising at least means for determining the components X_{i,ref} of the reference matrix, means for calculating the deviations e(m) from the reference situation and means for merging the data.

Finally, the invention relates to a medical assistance device comprising a monitoring device as described above.

### BRIEF DESCRIPTION OF THE FIGURES

Further objects, characteristics and advantages of the invention will become clearer in the following description, made with reference to the attached figures, in which:

FIG. 1 is a schematic diagram illustrating certain steps in the detection method according to the invention, from the extraction of data from different physiological signals to the merging of said data;

FIG. 2a shows the performance of three detectors for a subject SU1: the first figure from the left illustrates the performance (AUC) of an independent detector exploiting the electroencephalographic signals S₁ alone, the second figure from the left illustrates the performance (AUC) of an independent detector exploiting the respiratory signals S₂ alone and the third figure from the left illustrates the performance (AUC) of a detector exploiting the merged distances d_{fusion}, the fourth figure from the left (and therefore the figure furthest to the right) illustrates the curves ROC of these three detectors;

FIG. 2b differs from FIG. 2a in that performance is illustrated for a subject SU2;

FIG. 3 illustrates the effects of a filtering (smoothing) step on the performance of different detectors, in particular an independent detector using the respiratory signals S2 alone, an independent detector using the electroencephalographic signals S1 alone and a detector using the merged distances d_{fusion} (in this order, reading from left to right).

### DETAILED DESCRIPTION OF THE INVENTION

### Description of the General Mode of the Detection Method According to the Invention

The invention relates to a method for detecting a change in a patient's physiological condition. The patient's physiological condition is characterised by an assembly of functions and reactions of the patient, also referred to as physiological parameters. In the following, a distinction will be made between a reference physiological condition and a modified physiological condition, the latter condition characterising a change in the patient's physiological condition compared to his reference physiological condition. A change in the patient's physiological condition is considered to be a change in his physiological condition in relation to a reference physiological condition of the patient, when this change is due to at least one homeostatic change defined a few paragraphs later.

The reference physiological condition is not necessarily the physiological condition of the patient when he is healthy, i.e. the physiological condition in which the patient is when his functions and reactions are normal and/or the patient has no pathology. The reference physiological condition is the observed physiological condition of the patient from a given moment, whether the patient is in good or poor health, the observation being associated with a certain number of measurements, themselves associated with signals which will be described in more detail later.

The patient's modified physiological condition is not necessarily the patient's physiological condition when ill, i.e. the physiological condition in which the patient finds himself when his functions and reactions are abnormal and/or the patient has one or more pathologies. A patient's modified physiological condition corresponds to a physiological condition that differs from the reference physiological condition. This occurs when at least one or, in practice, several of the patient's functions or reactions deviate from the functions and/or reactions exhibited by the patient in the reference physiological condition. In this case, we speak of homeostatic modification.

A change in physiological condition does not therefore lie in the simple fact that the healthy patient becomes ill or vice versa, but lies in any change that is likely to take him from the reference physiological condition to another condition following a homeostatic modification. In other words, in the context of the invention, the patient's physiological condition does not presume whether or not the patient is healthy.

In the context of the invention, the reference physiological condition is associated with a matrix X_{ref}, referred to as the reference matrix, constructed from a plurality of components X_{ref,i} (i=[1 . . . N+1], N integer). Each component X_{ref,i} of the reference matrix X_{ref} is associated with a measurement of given dimension, the elements of which are the various physiological signals of the patient in the reference physiological condition. This reference physiological condition is associated with a reference period W₀. The dimensions of the reference matrix therefore depend on: 1) the number of physiological signals measured independently of the type of physiological signal to which the measured signal is actually assigned and, 2) the number of instants at which the measurements of the physiological signals are subsequently performed, i.e. the measurements performed outside the reference physiological condition which, for its part, is associated only with the reference period W₀.

With regard to the point 1) above, a component X_{ref,i} of the reference matrix may in particular be associated with spatio-temporal measurements, in particular when the assessment of the physiological parameter requires measurements to be performed in different regions of the body or of an area of the patient's body as a function of time (e.g. different electroencephalographic signals or different muscle signals recorded simultaneously). Such measurements are frequently carried out to assess the patient's cortical or muscular activity. They involve a series of electrodes, and if necessary needles, placed in appropriate areas of the patient's body in order to map, and therefore spatially represent, the evolution in the physiological parameter concerned. For example, electroencephalographic measurements involving 10 electrodes are therefore associated with 10 different physiological signals, one physiological signal being assigned to each electrode. If, in addition, these measurements are implemented at 8 different times, independently of any segmentation, the component X_{ref,i} of the reference matrix associated with the electroencephalographic measurements is a matrix of dimension 10×8. However, this does not presume the dimensions for the other components of the matrix X_{ref} which also comprises other signals X_{ref,i}.

With regard to the point 2) above, it is important to remember that while the invention seeks to establish correlations between the different types of homeostatic changes, and it is therefore essential, as will be seen below, that the measurements of the different types of physiological signals are performed in parallel, i.e. simultaneously, at least over a common time window, this does not mean that the time window over which the measurements are taken must be of the same length (number of samples or segments) for all the physiological parameters measured. The practitioner may wish to observe the evolution in one or more physiological parameters for longer than others. The invention remains within its scope as soon as the method is implemented with at least two types of physiological signals.

In any case, the reference matrix Xᵣe comprises as many components X_{ref,i} as there are types of physiological signal observed. In the invention, there are i=[1 . . . N+1] types of physiological signals measured.

In a first step A) of the detection method according to the invention, electroencephalographic measurements of the patient are performed in M time segments of an observation window. The electroencephalographic (EEG) measurements are used to measure the electrical activity of the patient's brain and take the form of recordings.

The observation window is a temporal observation window. It defines a time interval during which the measurements are performed at instants whose frequency can be predetermined. The M time segments define time sub-intervals of the observation window during which the continuous or discrete measurements of the electroencephalographic signals S₁ are performed. Each of the M time segments is assigned a duration noted W. If the observation window is the reference period defined above, each segment of the reference period is likewise assigned a duration noted W_{0,m}.

We define p instants at which the electroencephalographic measurements are carried out in a time segment m of said observation window. In addition, n paths are defined via which the patient's electroencephalographic measurements are performed. In practice, the patient is equipped with a headset comprising n electrodes suitably positioned on the patient's head to measure the electroencephalographic signals S₁, each electrode being associated with a path. A matrix of dimension n x p is generated for each time segment M, with the elements of the matrix corresponding to spatio-temporal measurements. We will denote X_{1,m}, m∈[1 . . . M] and M integer, the M measurement matrices thus generated.

Simultaneously, still during the first step A) of the method according to the invention, measurements are performed of at least one other type of physiological signal from the patient. As mentioned previously, the method described in this invention is a multimodal method for detecting a change in physiological condition. To this end, the method described in the invention is based on the simultaneous analysis of several of the patient's physiological parameters. It is sufficient for at least one other type of physiological parameter, other than the cortical activity (EEG), to be observed for the method to be considered a multimodal method. The other physiological signal or signals measured are electromyograms, the respiratory flow or rate, chest distension or breath sounds, electrocardiograms (ECG), electrodermal measurements or movement measurements. This is by no means limitative and other physiological parameters useful to the person skilled in the art for characterising the patient's physiological condition can be measured.

For the purposes of the invention, N are defined as the other types of physiological signals S_{N+1}, N≥1, measured other than the electroencephalographic signals S₁. There are therefore N+1 types of physiological signals from the patient which are measured simultaneously over at least one common observation window. As with the electroencephalographic signals S₁, measurements of the patient's other physiological signal or signals are taken in the M time segments of the observation window. For each time segment m, N other measurement matrices X_{i=2 . . . N+1,m} are generated. The dimensions of each matrix X_{N,m} depend on the type of physiological parameter under consideration.

Sub-steps B1), B2) and C1) described in the following relate to processing applied to the electroencephalographic signals S₁ according to a particular, i.e. non-limiting, implementation of the present invention. This implementation concerns the case where the electroencephalographic signals S₁ are processed in Riemannian geometry. Reference may be made in particular to the document WO 2013/164462 A1 which describes such steps. It should be noted that any other measured signal, i.e. other than the electroencephalographic signals S₁, for which this treatment is more appropriate may be subject to the treatments described in these steps.

According to a particular implementation, during an optional second step B), and more precisely a first sub-step B1), each signal of the matrix X_{1,m} is centred and filtered in Q predetermined frequency bands to obtain MxQ filtered measurement matrices X_{1,m,q}q∈[1 . . . Q]. Recall that the measurement matrices X_{1,m} were obtained by electroencephalographic measurements. The operations of sequential filtering and centring of measurement matrices have the usual mathematical meaning given to this type of operation. Advantageously, note that the measurement matrices X_{1,m} are centred and filtered in five frequency bands corresponding to the frequencies conventionally used for electroencephalography, namely 1-4 Hz, 4-8 Hz, 8-12 Hz, 12-24 Hz and 24-48 Hz. This allows only information from the regions of interest in the patient's brain to be retained, with each frequency range associated with one or more types of patient movement. For example, in the case of a plurality of electromyograms measured at different locations in the diaphragm, it would then have been appropriate to select frequency bands specific to the muscle dynamics of the diaphragm.

Still in the optional second step B), a second sub-step B2) consists of determining normalised spatial covariance matrices from the following formula:

$${C}_{m , q} = \frac{{X}_{1 , m , q} {X}_{1 , m , q}^{T}}{trace ( {X}_{1 , m , q} {X}_{1 , m , q}^{T} )} \qquad ( 1 )$$

where C_{m,q} are the normalised spatial covariance matrices, X_{1,m,q} are the filtered measurement matrices and X_{1,m,q}^{T} are the corresponding transposed matrices. The spatial covariance matrices C_{m,q} characterise the synchronisation of cortical activities over time. They comprise local synchronisations in the elements of the diagonal and remote synchronisations outside the diagonal, which are then the characteristics of the dynamics of the neural network.

According to a particular implementation, during a third step C), more precisely a first sub-step C1) of the third step, for each time segment m of the reference period W₀, Riemannian distances d_{1,r}(m) are determined between each normalised spatial covariance matrix C_{m,q} and the component X_{ref,1} of the reference matrix associated with the electroencephalographic measurements. More precisely, for each time segment m of the reference period W₀, the Riemannian distance d_{1,r}(m) is calculated between each normalised spatial covariance matrix C_{m,q} calculated in the time segment m, of duration W, at a time t:t+W and the component X_{ref,1} of the reference matrix calculated in the reference period W₀. To this end, in each frequency band, prototype matrices PR_{q,r}, where ∈[1 . . . R] are determined from the distribution of the spatial covariance matrices C_{m,q}. Each prototype is a representative of a subclass of the synchronisation, and is here estimated by a Karcher mean of the neighbourhood reference spatial covariance matrices C_{m,q}. It should be noted that in the case of a single prototype, this corresponds to the average covariance matrix for the entire reference period.

A procedure for calculating the prototypes PR_{q,r} according to a particular implementation, and therefore non-limiting, is described below and is applied for each frequency band. In this particular implementation, the calculation is based on the dynamic cluster algorithm (E. Diday, 1971) adapted to the Riemannian metric. Note that in signal processing, we usually use the classical Frobenius norm to define distances between covariance matrices (which are, by definition, Hermitian matrices defined as positive). This approach assumes a normed vector space with zero curvature. However, the space of the positive Hermitian definite matrices is more like a metric space with negative curvature. This particular implementation preferably uses the tools of Riemannian geometry to manipulate the covariance matrices. In this framework, the distance between two matrices corresponds to the geodesic in the space generated by their Hermitian property, and the average of the covariance matrices no longer corresponds to an arithmetic mean as conventionally, but to a geometric mean.

```
1: procedure
  C is an array of M covariance matrices.
  Q is the number of prototypes.
  M_{R} is the Karcher mean of an assembly of matrices
2: k:=M
3: eps:= 10⁻⁸
4: kk =random (k)
5: PR^{j}:=C_{kk(j);} j:=1:Q; PR_new^{j}:=0
6: as long as abs (PR_new^{j}) - PR^{j}) > eps
7: PR_new^{j}: = PR^{j};
8: for i:= 1 to k
9: label (i):= arg min _{j=1:Q} [dist (PR^{j}, Cⁱ)]
10:    end for
11: calculation of the Karcher mean for each partition PR
12:    for j:= 1 to Q;
13:    kj= [label (1:k): = j]; PR^{j} = M_{R} (C^{kj})
14:    end for
15: end as long as
16: return PR
```

According to this particular implementation, the distance dist, i.e. referred to as d_{i,r}, between the covariance matrices is the following Riemannian distance: if P₁, P₂ are two matrices, then:

$$dist ( {P}_{1} , {P}_{2} ) = {\{ {\sum }_{k = 1}^{K} {ln}^{2} ( {λ}_{k} ) \}}^{1 / 2} \qquad ( 2 )$$

where dist (P₁, P₂) is the Riemannian distance between the prototype matrices P₁ and P₂, the λₖ are the K eigenvalues of the joint matrix P₁⁻¹P₂.

The Riemannian distance dist verifies the three properties of a distance, namely the symmetry, the separation and the triangle inequality.

In one particular implementation, the average can be calculated using a gradient descent procedure that converges rapidly (Pennec et al. 2006):

1: procedure
C is an array of M covariance matrices.
2: CM:= C(1); d=0;
3: the average is initialized with the first value in the table.
4: e:= 10⁻⁸;
5: as long as d > e
6: W:= 0;
7: for i = 1 to M
8: W:= W + log_{CM} (C(i));
9: sum the tangent vectors in the tangent space
10: end for
11: W: = W/M;
12: we return to the variety with the exponential card and repeat
13: CM_new:= exp_{CM} (W): distance between 2 successive
iterations
14: d = dist (CM_new, CM)
15: CM=CM_new
16: end as long as
17: M_{R} = CM
18: return M_{R};

where the operators satisfy:

$${exp}_{CM} ( W ) = C {M}^{1 / 2} exp ( {CM}^{- \frac{1}{2}} W {CM}^{- \frac{1}{2}} ) {CM}^{\frac{1}{2}}$$

$${log}_{CM} ( W ) = C {M}^{1 / 2} log ( {CM}^{- \frac{1}{2}} W {CM}^{- \frac{1}{2}} ) {CM}^{\frac{1}{2}}$$

Subsequently or simultaneously, in a second sub-step C2) of the third step C, statistical distances d_{i,s}(m) are calculated between measured signal or signals X_{i,m} and the component or components X_{ref,i} of the reference period W₀. More precisely, for each time segment m, the statistical distances dᵢ(m) are calculated between one or more signals X_{i,m} measured in the time segment m, of duration W, at a time t:t+W and the component X_{ref,i} of the reference period W₀. The signal or signals concerned are those which have not been processed in the Riemannian geometry for whatever reason, for example because the Riemannian geometry is not suitable or does not prove to be suitable. In this particular implementation, a distance dᵢ therefore corresponds to either a Riemannian distance d_{i,r}(m) or to a statistical distance d_{i,s}(m), taking into account electroencephalographic signals S₁ and other signals. Statistical distances are calculated using the conventional mathematical processing.

It should be noted that steps B) and C) can be implemented without using the measurements of the electroencephalographic signals S₁ in the Riemannian geometry. The use of the electroencephalographic signals S₁ in the Riemannian geometry is easier because it allows to facilitate the measurements carried out at different points on the patient's head. However, this is not compulsory, even though this method gives better results. An alternative to the Riemannian distance between covariance matrices is the Euclidean distance between the matrices (the Frobenius norm of the algebraic difference of two matrices). Another alternative would be to measure distances between the signals EEG directly, without necessarily using covariance matrices. For example, we could use Hellinger's statistical distances, or Bhattacharyya's distance (Basseville, M. (1989). Distance measures for signal processing and pattern recognition. Signal processing, 18(4), 349-369).

Other methods described in the documents FR 2 903 314 A or US 2004/0254493 A1, already cited in the preamble to the description, can also be used to exploit such signals. The document FR 2 903 314 A describes another method for processing electroencephalographic signals, in which the distances correspond to an electroencephalographic potential. In the document US 2004/0254493 A1, the distances correspond to the differences between a maximum segment EEG power and a minimum segment EEG power, throughout the respiratory cycle. This is important because the acquisition of the electroencephalographic signals S₁ may not be available, or may contain numerous artefacts.

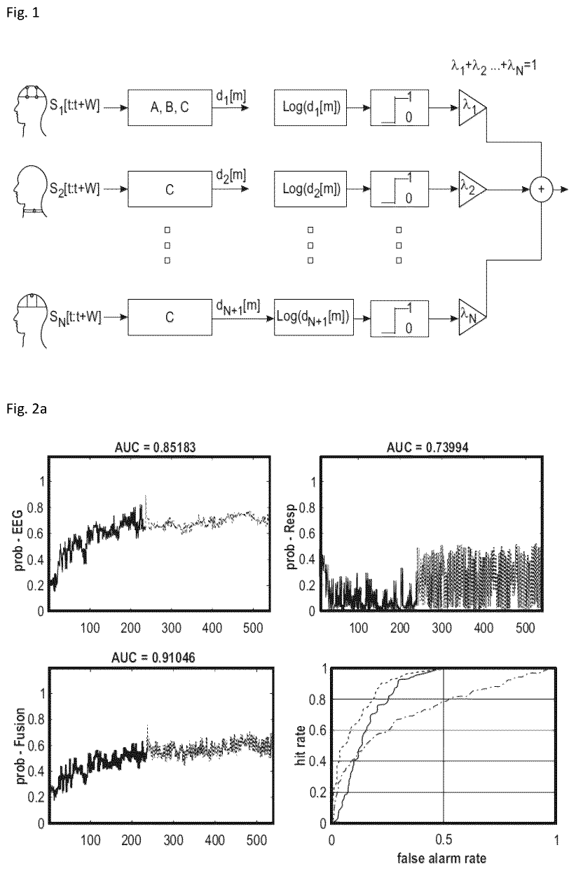

More generally, the distances can be calculated by any other method known to the prior art. In any case, a “distance” quantifies the statistical difference between two values of a physiological parameter. In other words, the distance is the mathematical expression of the change in a homeostatic constant at a given moment and its value in the reference period, which can be associated with the occurrence of a physiological change in the patient. To determine the merged distances d_{fusion}(m), as described in the remainder of this description, it is sufficient to determine the distances between each matrix of measurements performed in the time segments m and the component X_{ref,i}(i=[1 . . . N+1]) of the associated reference matrix. Consider only the signal S₂ in FIG. 1 of this application. A considered classifier calculates the distance between a variable/value measured at a given time, and the distribution of the same variable/value calculated in the reference period. More specifically, it could be a one-class SVM (Support Vector Machine) classifier or a classifier based on the Mahalanobis distance. The classifiers used to calculate the distances are therefore not restrictive.

The invention therefore does not lie in the methods used to calculate the distances, or even in the fact of using distances to merge data from different measurements, but in the very fact of merging data, as will be seen in more detail below. Step C) involves determining distances dᵢ(m) between each measured signal X_{i,m} and the reference period component X_{ref,i}, for each time segment m of the reference period W₀, where the distances dᵢ(m) may be Riemannian distances d_{i,r}(m) and/or statistical distances d_{i,s}(m) and/or any other type of distance, for example of the type seen previously.

With reference to FIG. 1, we now describe a specific implementation used to merge the data obtained from the Riemannian and statistical distances. The data merging carried out here makes the detection of changes in physiological condition more qualitative, more reliable and more precise. In addition, this merging allows us to better characterise the patient's physiological condition.

According to a particular implementation, in a fourth step D) of the method, the distances dᵢ(m) calculated in step C) are transformed using a log d(m) function. The aim of this step is to transform the Riemannian and statistical distances so that their variance is stabilised and their values lie between 0 and 1. In fact, significant deviations can be observed between the distances calculated within the different time segments m, regardless of the type of distance in question, i.e. statistical distance or Riemannian distance. We therefore need a better mathematical representation of the relative variations between the different distances calculated in the M time segments m, for each of the Riemannian and statistical distances. In a preferred embodiment, the aim is to make the distributions closer to the Gaussian distribution. However, the use of a Gaussian distribution is not obligatory and other types of distribution within the reach of the person skilled in the art can be envisaged insofar as they allow the variances of the measured data to be stabilised. At the end of step D), the logarithms log(dᵢ(m)) of the above distances are obtained.

According to this particular implementation, during a fifth step E), in particular a first sub-step E1) of the fifth step E), the precision of the values obtained at the end of step D) is chosen by selecting a reference quantile u₀ from all the values of the reference period W₀. In other words, during this first sub-step E1), the reference quantile u₀ is chosen to be applied to all the values measured during step D) by selecting it from all the values of the reference period W₀.

In practice, the appropriate quantile is chosen firstly as a function of the number of points in the distribution and secondly as a function of the accuracy required to describe the distribution, i.e. to describe all the values measured in the reference period W₀. Other criteria such as the number of outliers or the number of available data can also be taken into account to determine the reference quantile u₀. On an empirical basis, the present inventors have demonstrated the relevance of the ninth decile, i.e. the 90% quantile, as the reference quantile u₀ for obtaining the desired accuracy while taking into account the parameters of the distribution, in this case a Gaussian distribution (size, outliers, etc.). Selecting the reference quantile u₀ therefore does not necessarily involve additional analysis or calculation steps, since it is perfectly possible to implement the first sub-step E1) with the empirical value mentioned above. In other words, the reference quantile u₀ can be predetermined/preselected. For the sake of completeness, the first sub-step E1) could be implemented before step E), once the data for the reference period W₀ is known.

Still according to this particular implementation, in a second sub-step E2), a scalar variable p_{i=1, . . . ,N+1}(0<_{i=1, . . . ,N+1}<1) dependent on the reference quantile u₀ is associated with each distance dᵢ(m). In other words, in this second sub-step E2), the quantile u₀ preselected or selected in the first sub-step E1) is used to make scalar assignments. Each of the log(dᵢ(m)) calculated at the end of step D) is assigned a scalar variable p_{i=1, . . . ,N+1} according to the (pre)selected reference quantile u₀. This step ensures that all the data associated with the physiological parameters observed can be classified in relation to each other, regardless of the type of data to which they were originally linked, grouped by type and merged (in step F in particular). At the end of step E), we therefore have a classifier in which the data obtained from the various physiological signals measured can be compared, classified and merged.

In a preferred embodiment, the scalar variables p_{i=1, . . . ,N+1} (0<p_{i=1, . . . ,N+1}<1) are calculated from the following transformation:

$${p}_{i = 1 , … , N + 1} ( m ) = \frac{1}{1 + {e}^{{u}_{0}} - log ( {d}_{i = 1 , … , N + 1} ( m ) )} \qquad ( 3 )$$

where p_{i=1, . . . ,N+1} are the scalar variables, u₀ is the reference quantile, d_{i=1, . . . ,N+1}(m) are the Riemannian distances and the statistical distances.

Calculated in this way, the scalar variables p_{i=1, . . . ,N+1} are therefore presented in the form of probabilities. The use of scalar variables p_{i=1, . . . ,N+1} in such a form facilitates their mathematical processing and therefore simplifies the implementation of the detection method according to this particular implementation.

In a sixth step F), the data obtained in step E) is merged by performing a weighted sum of the variables p_{i=1, . . . ,N+1} to obtain the resulting distances d_{fusion}(m). In other words, for each of the M time segments m, a distance d_{fusion}(m) is obtained by merging the data obtained at the end of step E) with a weight, i.e. a weighting coefficient.

One advantage of segmenting the observation window into M time segments m is that the data from the different types of physiological signal are well correlated.

Moreover, while data merging as such allows to reduce the amount of data and therefore provide a greater algorithmic efficiency, it is also very effective in that it allows a change in the patient's physiological condition to be deduced very precisely. In fact, each merged distance d_{fusion}(m) takes into account the different types of physiological signals measured and allows the information contained in these different signals to be integrated. Instead of superimposing the measurements, possibly from different sensors, of the same type of physiological signal, as has been done in the prior art, the measurements of different types of physiological signals are superimposed and coupled. In the context of the invention, the fact of “merging” the measurements does not refer to the simple fact of matching the measurements, i.e. considering them in pairs, threes, etc. The fact of “merging or combining” must be understood as the fact of generating a result, in this case d_{fusion}(m), by mathematical transformation of the data from the measurements, the data being the distances dᵢ(m) previously calculated. Any detection of a change in the patient's physiological condition is therefore the result of a combination/merging of data from the various physiological parameters. A change in the patient's physiological condition established on the basis of the method according to the invention means that several homeostatic modifications, at least 2, have therefore taken place. It is therefore more representative of the patient's general condition.

Performing weighted sums of the scalar variables p_{i=1, . . . ,N+1}(0<p_{i=1, . . . ,N+1}<1), i.e. summing the scalar variables p_{i=1, . . . ,N+1}, previously assigned a weighting coefficient, substantially improves the accuracy of the merged distances d_{fusion}(m). This is because it is possible to assign less weight to data from signals that are the most contaminated, for example by noise, artefacts, etc., and on the contrary to assign a greater weight to the signals of better quality. In this case, the weighting coefficient is defined on the basis of an a posteriori criterion.

It is also possible to define a weighting coefficient based on an a priori criterion. For example, it is possible to assign a weighting coefficient according to the importance of the signal Sᵢ with which it is associated in determining the change in the patient's physiological condition, i.e. according to the importance of the physiological parameter observed in the change in the patient's physiological condition. An a priori criterion has the advantage of making the calculation of d_{fusion}(m) more objective than an a posteriori criterion. However, there is nothing to prevent the use of a weighting coefficient resulting from an a priori criterion and a posteriori criterion. The type of weighting coefficient selected is not limitative in the context of the present invention. Moreover, it is possible to assign an identical weighting coefficient to all the distances the scalar variables p_{i=1, . . . ,N+1} calculated, which amounts to giving identical importance to all the signals measured.

All the advantages described above work together to improve the detection of a change in the patient's physiological condition. This makes the detection method described in the invention all the more reliable and efficient compared with the methods known up to now in the prior art.

According to a preferred implementation of the present invention, d_{fusion}(m) is calculated as follows:

$${d}_{fusion} ( m ) = {\sum }_{i} {λ}_{i} {p}_{i = 1 , … , N + 1} ( m ) \qquad ( 4 )$$

where λᵢ is the weight assigned to each variable p_{i=1, . . . ,N+1}. The scalar variables p_{i=1, . . . ,N+1}, for example, can be calculated using the formula (3) described above. The above advantages are not repeated. However, we specify that it is preferable for the sum of the weighting coefficients λᵢ to be such that λ₁+λ₂+ . . . +λ_{N}=1. The purpose of this is to simplify the mathematical processing of the data in this particular implementation of the detection method according to the invention.

Once the d_{fusion}(m) distances have been calculated, a deviation e(m) from the physiological reference condition is determined as a function of the d_{fusion}m) distances in a seventh step G). As we have already seen, this is made possible by the fact that the reference physiological condition, to which the reference matrix X_{ref} with components X_{ref,i} is associated, is already known. With regard to the determination of the deviation e(m) from the physiological reference condition, it should also be noted that not every deviation is necessarily considered to reflect a change of condition. In fact, a threshold Θ can be defined at which a deviation e(m) from the reference physiological condition is considered sufficiently significant to consider that there is indeed a change in the patient's physiological condition. The deviation e(m) which is calculated is that between the merged distances d_{fusion}(m) at the instants t:t+W and the merged distances.

According to a particular implementation of the method according to the invention, prior to step G, the merged data is filtered. This filtering step consists of smoothing using a moving average over the L last time segments m of the observation window (1<L<M) applied to the resulting distances d_{fusion}(m). The aim of smoothing is to further reduce the number of false alarms from the respiratory assistance device by reducing the irregularities generally caused by the signal attenuation. Preferably, this smoothing step is implemented by applying the following relationship to d_{fusion}(m):

$${d}_{fusion}^{L} ( m ) = \frac{1}{L} {\sum }_{i = 0}^{L - 1} {d}_{fusion} ( m - i ) \qquad ( 5 )$$

The value of the merging distance calculated in this way significantly increases the performance of the method. Smoothing the merged distances d_{fusion}(m) provides a better overall classification by reducing their variability, for all M time segments.

Advantageously, the L last segments are chosen so that the moving average is calculated taking into account the signals measured only with a delay of between 10 seconds and 1 minute, preferably between 30 seconds and 1 minute, relative to the start of the observation window. By lengthening the time window over which the windows are smoothed, in particular up to approximately 1 minute, it is possible to considerably improve the performance of the detection method according to this particular implementation of the invention, as will be seen in more detail below.

Note that the detection method according to the invention can be implemented using any data processing other than that described above in relation to steps B) to F). In fact, in the method described in the invention, the important thing is to merge the data from the measurements performed from the different types of signal. In addition, if in a preferred embodiment this merging is implemented by performing a weighted sum of the data obtained at the end of steps B) to E) in order to obtain resulting distances d_{fusion}(m), another merging method based on data other than distances may be envisaged. In other words, the specific implementation previously described with reference to steps B) to F) is proposed for the sole purpose of facilitating the understanding of the invention. It might be possible to merge the data from the various measurements using methods known to the person skilled in the art, such as Bayes probability-based methods, Dempster-Shafer belief theory, transferable belief models, Dubois and Prade possibility theory, and so on.

### Practical Application of the Method for Detecting a Change in a Patient's Physiological Condition

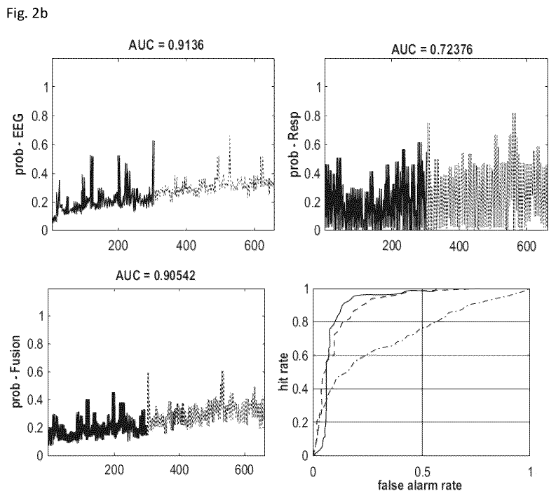

In this example of embodiment, the method for detecting a change in physiological condition according to the invention is implemented for two subjects SU1 and SU2 on the basis of measurements of electroencephalographic signals S₁ and measurements of a respiratory signal S₂ from each subject. In this example of embodiment, the method has been implemented as described above, using successive steps A to G according to a particular implementation of the method according to the invention. FIGS. 2a and 2b illustrate the performance obtained with the detection method according to the invention for a subject SU1 (FIG. 2a) and a subject SU2 (FIG. 2b).

In each of FIGS. 2a and 2b, the first figure illustrates the Riemannian distances d_{1,r} estimated from the electroencephalographic signals S₁ alone, while the second figure illustrates the statistical distances d₂ estimated from the respiratory signal S₂ alone. In other words, these first two figures show the performance associated with an independent detector based on the analysis of a single type of signal from among the aforementioned types of signal. On each curve in the figures, there is a first segment m1 and a second segment m2 in which Riemannian and statistical distances have been calculated (corresponding to the two physiological conditions of a patient). In each of FIGS. 2a and 2b, the third figure illustrates the distances d_{fusion} obtained after merging log d_{1,r} and log d_{2,s} according to formula (4) above. We have opted for the 90% quantile—the ninth decile—to define precision. Here, the weighting coefficients are identical λ₁=λ₂=0.5.

Each figure (sub-figure) shows a value corresponding to the Area Under the Curve (AUC), which can be used to monitor the detection performance. For subject SU1, the AUC values associated with the distances calculated from the electroencephalographic signals S₁ alone, from the respiratory signals S₂ alone and from the d_{fusion} distances resulting from the merging of these two types of data are equal to 0.85, 0.74 and 0.91 respectively. The net increase in AUC after data merging corresponds to a net improvement in performance. For the subject SU2, the AUC values associated with the distances calculated from the electroencephalographic signals S₁ alone, from the respiratory signals S₂ alone and from the d_{fusion} distances resulting from the merging of these two types of data are equal to 0.91, 0.72 and 0.91 respectively. In this case, although the increase in AUC was not as significant as that seen for the subject SU1, there was an improvement in the quality of the merged data compared with the unmerged data.

In each of FIGS. 2a and 2b, the fourth figure illustrates the receiver operating characteristic (ROC) for each subject. Each figure shows three curves, from lightest to darkest, corresponding to the ROC curve associated with the respiratory signal S₂ alone, the ROC curve associated with the electroencephalographic signals S₁ alone and the ROC curve associated with the distances d_{fusion} resulting from the merging of the data from these two types of measurement. It can be seen that the false alarm rate increases less rapidly with a detector using the merged distances d_{fusion} compared with what is measured by independent detectors, i.e. the detectors using the electroencephalographic signals S₁ alone or the respiratory signal S₂ alone. It can be seen that for the subject SU2, the independent detector using the electroencephalographic signals S₁ alone nevertheless showed comparable performance to that of the detector using the merged data.

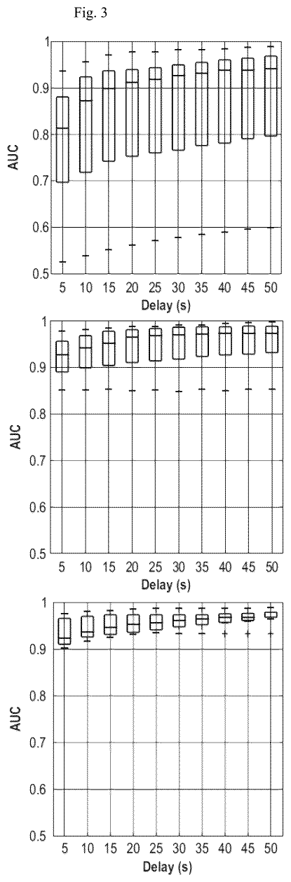

As mentioned in the general description, it is also possible to improve the performance of the detectors by filtering the data. While, in the method according to the invention, this preferred filtering step is applied to the merged data, it can also be applied to unmerged data. The inventors have implemented this smoothing to merged and unmerged data in order to demonstrate the performance of the merging. This is illustrated in FIG. 3.

An improvement in the performance of each of the detectors can already be seen by performing a smoothing on the data obtained from 15 seconds onwards (i.e. with a 15 second delay). As can be seen in FIGS. 3a, 3b and 3c respectively, the AUC values associated with each detector increase as the delay increases. While an improvement in performance was clearly seen for the detectors using the respiratory signal S₂ alone and the electroencephalographic signals S₁ alone, it was even more marked for the detector using the merged data. The results are remarkable, as shown in FIG. 3c).

### Description of a General Embodiment of a Patient Monitoring Device According to the Invention

First of all, it should be noted that by patient monitoring device we mean any device as described in the following where it can measure at least two physiological parameters of a patient, i.e. at least two functions and/or reactions of the patient.

The invention also relates to a patient monitoring device allowing a detection method as previously described to be implemented. Advantageously, the monitoring device is equipped with a processor allowing it to perform tasks, including data processing, and to communicate with other equipment to which it is linked and/or connected.

In order to implement the method according to the invention, the patient monitoring device comprises means for measuring electroencephalographic signals S₁. These measuring devices typically comprise a helmet equipped with n electrodes positioned appropriately on the patient's head. These means may also comprise any other equipment allowing electroencephalographic measurements to be taken. The helmet is connected to the monitoring device and communicates all the data measured by the electrodes. The monitoring device is able to receive and process the information received from the helmet and, where necessary, adapt the ventilatory assistance provided to the patient.

The monitoring device also comprises means for measuring the cardiac activity and/or the respiratory activity and/or the muscular activity and/or the movement. In this respect, the monitoring device is linked and/or connected to the sensors and other diagnostic tools commonly used to measure the physiological parameters concerned. These sensors or other tools referred to may consist of cervical electrodes or accelerometers, depending on the type of physiological parameter being observed.

As previously mentioned, the monitoring device is able to perform data processing tasks. It comprises real-time signal processing means, in particular means for determining the components X_{i,ref} of the reference matrix, means for calculating the deviations e(m) from the reference situation and data merging means. In any event, it is suitable for implementing the method covered by the invention.

The invention also relates to a medical assistance device equipped with a monitoring device as previously described.

1. A method for detecting a change in the physiological condition of a patient relative to a reference physiological condition associated with a reference matrix X_{ref} with components X_{ref,i} (i=[1 . . . N+1], N integer) and with a reference period W₀, the method implementing the following steps in a loop:
A) in M time segments m of an observation window, performing measurements of electroencephalographic signals S₁ from the patient along n paths and at p times to generate M measurement matrices X_{1,m}(mϵ[1 . . . M], M integer) each comprising n*p samples, and simultaneously performing measurements of at least N other types of physiological signals S_{N+1}(N≥1) from the patient, different from the electroencephalographic signals S₁, to generate N other measurement matrices X_{i=2 . . . N+I,m},
C) for each time segment m of the reference period W₀, determining distances dᵢ(m) between each measured signal X_{i,m} and the component X_{ref,i} of the reference period,
D) transforming the distances dᵢ(m) calculated in step C) using a log dᵢ(m) function,
E) choosing the precision of the values obtained at the end of step D) by selecting a reference quantile u₀ from all the values of the reference period W₀ and associating with each distance dᵢ(m) a scalar variable p_{i=1, . . . , N+1}(0<p_{i=1, . . . , N+1}<1) depending on the reference quantile u₀,
F) merging the data obtained in step E) by performing a weighted sum of the variables p_{i=, . . . , N+1} to obtain the resulting distances d_{fusion}(m),
G) determining a deviation e(m) from the physiological reference condition as a function of the distances d_{fusion}(m).

2. The method according to claim 1, wherein in a step B) the electroencephalographic signals S₁ are centered and filtered in Q predetermined frequency bands to obtain MxQ filtered measurements matrices X_{I,m,q}(qϵ[1 . . . Q]) and MxQ normalised spatial covariance matrices are determined by the formula:
$${C}_{m , q} = \frac{{x}_{1 , m , q} {x}_{1 , m , q}^{T}}{trace ( {x}_{1 , m , q} {x}_{1 , m , q}^{T} )}$$
and in which, in step C), Riemannian distances d_{i,r}(m) are determined between each normalised spatial covariance matrix C_{m,q} and the components X_{ref,i} the reference matrix associated with the electroencephalographic measurements.

3. The method according to claim 1, wherein, during step C), statistical distances d_{i,s}(m) are determined between one or more measured signals X_{i,m}, other than the electroencephalographic signals, and the component or components X_{ref,i} of the reference period associated with it.

4. The method according to claim 1, wherein d_{fusion}(m) is calculated according to the following relationship:
$${d}_{fusion} ( m ) = {\sum }_{i} {λ}_{i} {p}_{i = 1 , … , N + 1} ( m ) , with$$
$${p}_{i = 1 , … , N + 1} ( m ) = \frac{1}{1 + {e}^{{u}_{0}} - log ( {d}_{i = 1 , … , N + 1} ( m ) )}$$
where λᵢ is the weight assigned to each variable p_{i=1, . . . , N+1}, the λᵢ verifying Σᵢλᵢ=1, u₀ is the reference quantile, d_{1,q}(m) are the Riemannian distances and d_{i>1}(m) are the statistical distances.

5. The method according to claim 4, wherein the weights λᵢ are selected according to the quality of the signal S₁ with which they are associated or according to the importance of the signal S₁ with which they are associated for determining a change in physiological condition in the patient.

6. The method according to claim 1, wherein a step of filtering the resulting distances d_{fusion}(m) is implemented prior to step G).

7. The method according to claim 6, wherein the filtering consists of a smoothing obtained by a moving average over the last L segments of the observation window (1<L<M) applied to the resulting distances d_{fusion}(m), said distance d_{fusion}(m) then being calculated according to the following relationship:
$${d}_{fusion}^{L} ( m ) = \frac{1}{L} {\sum }_{i = 0}^{L - 1} {d}_{fusion} ( m - i )$$

8. The method according to claim 7, wherein the last L segments are chosen so that the moving average is calculated taking account of the measured signals only with a delay of between 10 seconds and 1 minute, preferably between 30 seconds and 1 minute, relative to the start of the observation window.

9. The method according to claim 1, wherein said other physiological signals Sᵢ are cardiac activity and/or respiratory activity and/or muscular activity and/or a movement-related signal.

10. The method according to claim 1, further comprising a step during which each of the deviations e(m) is compared with a determined threshold Θ.

11. A device for monitoring a patient for the implementation of a method according to claim 1, comprising:
electroencephalographic signal measuring means for measuring the patient's electroencephalographic signals S₁,
means for measuring cardiac activity and/or respiratory activity and/or muscular activity and/or movement,
real-time signal processing means, said means comprising at least means for determining the components X_{i,ref} of the reference period, means for calculating the deviations e(m) from the reference situation and means for merging the data.

12. A medical assistance device comprising a monitoring device according to claim 11.
